(19) United States
(12) Patent Application Publication
(10) Pub. No.: US 2018/0368742 A1
(43) Pub. Date: Dec. 27, 2018

(54) BIOSENSOR FOR DETECTING GLUCOSE LEVEL, METHOD FOR MAKING THE BIOSENSOR, AND METHOD FOR DETECTING GLUCOSE LEVEL

(71) Applicant: HON HAI PRECISION INDUSTRY CO., LTD., New Taipei (TW)

(72) Inventor: HSIU-WEN CHIEN, New Taipei (TW)

(21) Appl. No.: 15/649,738

(22) Filed: Jul. 14, 2017

(30) Foreign Application Priority Data
Jun. 27, 2017 (TW) 106121343

(51) Int. Cl. A61B 5/145; A61B 5/1455

(52) U.S. Cl. CPC A61B 5/14532; A61B 5/1455

(57) ABSTRACT

A biosensor for detecting body glucose level includes a gel substrate. Glucose oxidase, peroxidase, and a color developing agent are dispersed in the gel substrate. The gel substrate defines a plurality of pores each having a diameter of about 50 nm to about 550 nm and the biosensor in contact with body fluids such as blood or tears changes color to indicate body glucose level.

### FIELD

The subject matter herein generally relates to health monitoring, and more particularly, to a biosensor for detecting glucose level, a method for making the biosensor, and a method for detecting glucose level using the biosensor.

### BACKGROUND

Monitoring glucose levels in blood regularly is very important in diabetes management. Body fluids, including urine and tears, are alternative sources for tracking the glucose levels. Tracking glucose levels in urine and tears is non-invasive and convenient, but the biosensor for monitoring the glucose levels in blood, urine, or tears is not sensitive enough. Improvement in the art is preferred.

### BRIEF DESCRIPTION OF THE DRAWINGS

Implementations of the present disclosure will now be described, by way of example only, with reference to the attached figures.

FIG. 1 is a flowchart of an exemplary embodiment of a method to make a biosensor for detecting glucose level.

FIG. 2 is a diagram of an exemplary embodiment of a biosensor for detecting glucose level.

FIG. 3 is a flowchart of an exemplary embodiment of a method for detecting glucose level.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean at least one.

The term “comprising,” when utilized, means “including, but not necessarily limited to”; it specifically indicates open-ended inclusion or membership in the so-described combination, group, series, and the like.

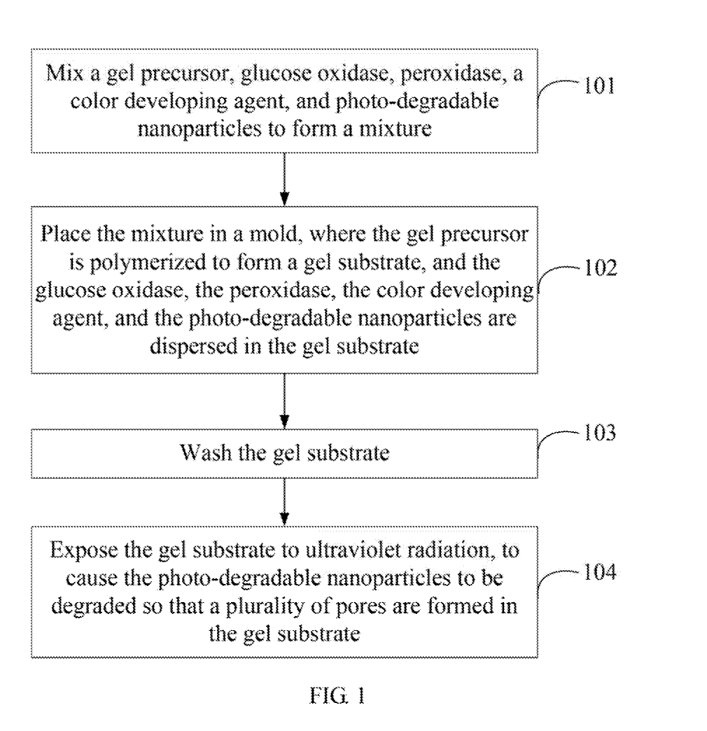

FIG. 1 illustrates a flowchart of an exemplary embodiment for a method to make a biosensor for detecting glucose level. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin at block 101.

At block 101, a gel precursor, glucose oxidase (GOx), peroxidase, a color developing agent (CDA), and photo-degradable nanoparticles are mixed to form a mixture. The photo-degradable nanoparticles are nanoparticles that can be degraded under ultraviolet radiation. The photo-degradable nanoparticles each have a diameter of about 35 nm to about 250 nm.

In at least one exemplary embodiment, the gel precursor has a mass percentage of about 58.2% to about 98% of a total mass of the mixture. The glucose oxidase has a concentration of about 62 units/g to about 158 units/g in the mixture. The peroxidase has a concentration of about 18 units/g to about 205 units/g in the mixture. The color developing agent has a mass percentage of about 1.86% to about 42% of the total mass of the mixture. The photo-degradable nanoparticles have a mass percentage of about 0.13% to about 8.6% of the total mass of the mixture.

In at least one exemplary embodiment, the gel precursor comprises hydrophilic monomers, a cross-linking agent, and an initiator.

The hydrophilic monomers can be selected from a group consisting of 2-hydroxyethyl methacrylate (HEMA), N,N′-dimethylacrylamide (DMA), methyl methacrylate (MMA), N-vinyl pyrrolidone (NVP), polyethylene glycol maleate (PEGMA), tris(trimethylsilyl)silane (TRIS), polydimethylsiloxane (PDMS), hydroxyethyl acrylate (HEA), hydroxypropyl methacrylate (HPMA), dimethylaminoethyl methacrylate (DMAEMA), methyl acrylate (MA), and any combination thereof.

The cross-linking agent can be selected from a group consisting of ethylene glycol dimethacrylate (EGDMA), trimethylolpropane trimethacrylate (TMPTMA), N,N′-methylenediacrylamide (MBAA), and any combination thereof.

The initiator can be a mixture of ammonium persulfate (APS) and N,N,N′,N′-tetramethylethylenediamine (TEMED).

In at least one exemplary embodiment, the hydrophilic monomers have a mass percentage of about 69.3% to about 99% of a total mass of the gel precursor. The cross-linking agent has a mass percentage of about 0.45% to about 12.8% of the total mass of the gel precursor. The initiator has a mass percentage of about 0.36% to about 19.5% of the total mass of the gel precursor.

In at least one exemplary embodiment, the peroxidase can be horseradish peroxidase (HRP).

In at least one exemplary embodiment, the color developing agent can be selected from a group consisting of potassium iodide, 3-hydroxy-2,4,6-tribromobenzoic acid (TBHBA)/4-aminoantipyrine (4-AAP), o-phenylenediamine (OPD), and tetramethylbenzidine (TMB).

The formation of the photo-degradable nanoparticles can be carried out by mixing photodegradable acrylate (PA, chemical structure

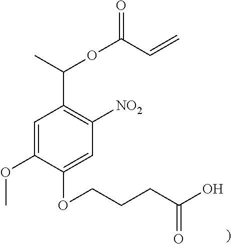 [SMILES: C=CC(=O)OC(C)c1cc(OC)c(OCCCC(=O)O)cc1[N+](=O)[O-]]

and polyethylene glycol dimethacrylate (PEGDMA) to form a water phase solution. Ammonium persulfate and N,N,N′,N′-tetramethylethylenediamine are mixed to form an oil phase solution. The oil phase solution is stirred, and the water phase solution is added in the oil phase solution being stirred to form water-in-oil (W/O) emulsion, wherein the photodegradable acrylate, the polyethylene glycol dimethacrylate, and the ammonium persulfate, and N,N,N′,N′-tetramethylethylenediamine are polymerized in the W/O emulsion to obtain the photo-degradable nanoparticles. Finally, the W/O emulsion is filtered to obtain the photo-degradable nanoparticles. In at least one exemplary embodiment, the oil can be coconut oil or olive oil. The water phase solution and the oil phase solution are in a ratio selected from 1:16 to 1:105 by volume.

The photo-degradable nanoparticles have o-nitrobenzylether groups (chemical structure:

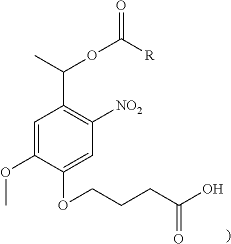

that can be degraded under ultraviolet radiation according to the following reaction:

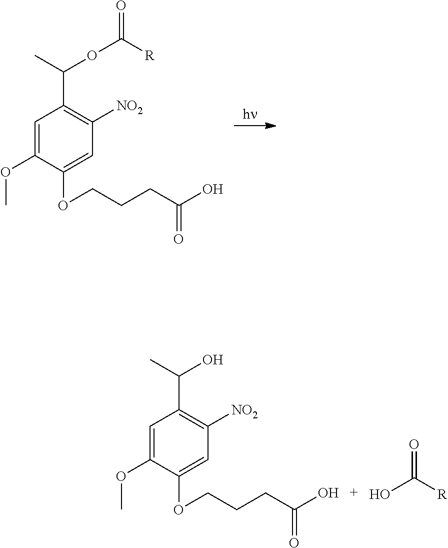

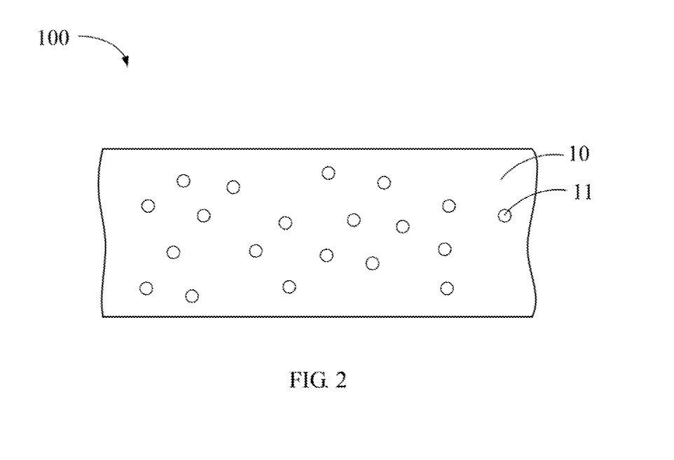

At block 102, referring to FIG. 2, the mixture is placed in a mold, where the gel precursor is polymerized to form a gel substrate 10, and the glucose oxidase, the peroxidase, the color developing agent, and the photo-degradable nanoparticles are dispersed in the gel substrate 10.

In at least one exemplary embodiment, the initiator is a mixture of APS and TEMED, which is not a photoinitiator or a thermal initiator. Thus, the mixture has no need to be heated or exposed to ultraviolet radiation, thereby preventing the glucose oxidase from losing its activity when heated and the photo-degradable nanoparticles from being degraded when exposed to ultraviolet radiation, during the formation of the gel substrate 10.

At block 103, the gel substrate 10 is washed.

In at least one exemplary embodiment, the gel substrate 10 can be washed in deionized water or a buffer solution.

At block 104, the gel substrate 10 is exposed to ultraviolet radiation, which causes the photo-degradable nanoparticles to be degraded so that a plurality of pores 11, each pore having a diameter of about 50 nm to about 550 nm, are formed in the gel substrate 10. Thus, the biosensor 100 is formed.

The biosensor 100 can be applied in a contact lens (for tears) or a wound dressing (for blood) to detect glucose level.

When the biosensor 100 contacts glucose in blood or in tears, the glucose reacts with oxygen in the ambient environment under the function of glucose oxidase to form hydrogen peroxide (H₂O₂). The hydrogen peroxide oxidizes the color developing agent under the function of the peroxidase, which causes the color developing agent to change from colorless to having a color. Thus, the biosensor 100 can display colors corresponding to the glucose level. Since the color of the biosensor 100 can be observed by a user, the glucose level can be determined by a comparison between the color of the biosensor 100 and a color reference card. The biosensor 100 is porous, which improves a contact area between the glucose and the biosensor 100. That is, the glucose can enter the biosensor 100 to fully react with the glucose oxidase, which improves the sensitivity of the biosensor 100. Take β-D-glucose for example, the reaction between the glucose and the biosensor 100 can be shown as follows:

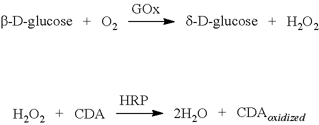

### Example 1

PA and PEGDMA in a ratio of 18:82 by weight were mixed to form a water phase solution. APS, TEMED and coconut oil in a ratio of 1:0.5:98.5 by weight were mixed to form an oil phase solution. The oil phase solution was stirred at 8000 rpm, and the water phase solution was added in the oil phase solution being stirred to form W/O emulsion (the water phase solution and the oil phase solution being in a ratio of 1:20 by volume). The W/O emulsion was filtered to obtain photo-degradable nanoparticles having a diameter of about 120 nm. HEMA, APS/TEMEA, and EGDMA in a ratio of 98:1:1 by weight were mixed to form a gel precursor. The gel precursor, GOx, HRP, potassium iodide, and the photo-degradable nanoparticles were mixed to form a mixture. In the gel precursor, the GOx had a concentration of 125 units/mL, the HRP had a concentration of 25 units/mL, the potassium iodide had a concentration of 6 mol/L, and the photo-degradable nanoparticles had a concentration of 3 mg/mL. The mixture was placed in a mold to form a gel substrate 10. The gel substrate 10 was washed in deionized water and exposed to ultraviolet radiation of 365 nm for 5 minutes, thus forming the biosensor 100. The biosensor 100 was immersed in a glucose solution having a concentration of 0.5 mol/L. Then, the biosensor 100 changes from colorless to blue. The glucose level can be determined by a comparison between the color of the biosensor 100 and a color reference card. A spectrometer was further used to confirm the same glucose level.

### Example 2

PA and PEGDMA in a ratio of 25:75 by weight were mixed to form a water phase solution. APS, TEMED and coconut oil in a ratio of 1:1:98 by weight were mixed to form an oil phase solution. The oil phase solution was stirred at 20000 rpm, and the water phase solution was added in the oil phase solution being stirred to form W/O emulsion (the water phase solution and the oil phase solution being in a ratio of 1:50 by volume). The W/O emulsion was filtered to obtain photo-degradable nanoparticles having a diameter of about 80 nm. HEMA, DMA, TRIPS, APS/TEMEA, and tmptma in a ratio of 27:18:52:1.6:1.4 by weight were mixed to form a gel precursor. The gel precursor, GOx, HRP, TBHBA/4-AAP, and the photo-degradable nanoparticles were mixed to form a mixture. In the gel precursor, the GOx had a concentration of 70 units/mL, the HRP had a concentration of 160 units/mL, the TBHBA/4-AAP had a concentration of 5 mg/mL and 20 mg/mL, and the photo-degradable nanoparticles had a concentration of 5 mg/mL. The mixture was placed in a mold to form a gel substrate 10. The gel substrate 10 was washed in deionized water and exposed to ultraviolet radiation of 420 nm for 22 minutes, thus forming the biosensor 100. The biosensor 100 was immersed in a glucose solution having a concentration of 0.05 mol/L. Then, the biosensor 100 changes from colorless to pink. The glucose level can be determined by a comparison between the color of the biosensor 100 and a color reference card. A spectrometer was used to confirm the same glucose level.

FIG. 2 illustrates an exemplary embodiment of a biosensor 100. The biosensor 100 can be applied in a contact lens or a wound dressing to detect glucose level. The biosensor 100 comprises a gel substrate 10. Glucose oxidase, peroxidase, and a color developing agent are dispersed in the gel substrate 10. The gel substrate 10 defines a plurality of pores 11 each having a diameter of about 50 nm to about 550 nm.

In at least one exemplary embodiment, the gel substrate 10 is made of hydrogel or silicone hydrogel.

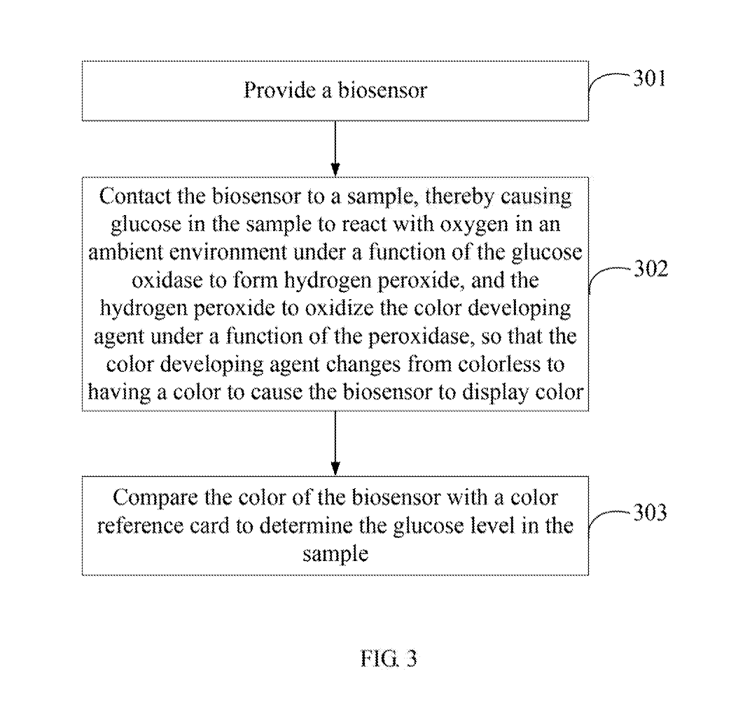

FIG. 3 illustrates a flowchart of an exemplary embodiment for a method for detecting glucose level. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin at block 301.

At block 301, referring to FIG. 2, a biosensor 100 is provided. The biosensor 100 comprises a gel substrate 10. Glucose oxidase, peroxidase, and a color developing agent are dispersed in the gel substrate 10. The gel substrate 10 defines a plurality of pores 11 each having a diameter of about 50 nm to about 550 nm.

At block 302, the biosensor 100 contacts a sample (blood or tears), thus causing the glucose in the sample to react with oxygen in the ambient environment under the function of the glucose oxidase to form hydrogen peroxide (H₂O₂). The hydrogen peroxide oxidizes the color developing agent under the function of the peroxidase. Thus, the color developing agent changes from colorless to having a color. Then, the biosensor 100 displays color.

At block 303, the color of the biosensor 100 is compared with a color reference card to determine the glucose level in the sample.

Depending on the embodiment, certain of the steps of methods hereinbefore described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A method for making a biosensor comprising:
mixing a gel precursor, glucose oxidase, peroxidase, a color developing agent, and photo-degradable nanoparticles to form a mixture, the photo-degradable nanoparticles having o-nitrobenzylether groups, the photo-degradable nanoparticles each having a diameter of about 35 nm to about 250 nm;
placing the mixture in a mold; wherein the gel precursor is polymerized to form a gel substrate, and the glucose oxidase, the peroxidase, the color developing agent, and the photo-degradable nanoparticles are dispersed in the gel substrate;
washing the gel substrate; and
exposing the gel substrate to ultraviolet radiation to cause the photo-degradable nanoparticles to be degraded so that a plurality of pores being formed in the gel substrate, and thereby forming the biosensor.

2. The method of claim 1, wherein the gel precursor has a mass percentage of about 58.2% to about 98% of a total mass of the mixture, the glucose oxidase has a concentration of about 62 units/g to about 158 units/g in the mixture, the peroxidase has a concentration of about 18 units/g to about 205 units/g in the mixture, the color developing agent has a mass percentage of about 1.86% to about 42% of the total mass of the mixture, and the photo-degradable nanoparticles have a mass percentage of about 0.13% to about 8.6% of the total mass of the mixture.

3. The method of claim 1, wherein the gel precursor comprises hydrophilic monomers, a cross-linking agent, and an initiator.

4. The method of claim 3, wherein the hydrophilic monomers are selected from a group consisting of 2-hydroxyethyl methacrylate, N,N′-dimethylacrylamide, methyl methacrylate, N-vinyl pyrrolidone, polyethylene glycol maleate, tris(trimethylsilyl)silane, polydimethylsiloxane, hydroxyethyl acrylate, hydroxypropyl methacrylate, dimethylaminoethyl methacrylate, methyl acrylate, and any combination thereof.

5. The method of claim 3, wherein the cross-linking agent is selected from a group consisting of ethylene glycol dimethacrylate, trimethylolpropane trimethacrylate, N,N′-methylenediacrylamide, and any combination thereof.

6. The method of claim 3, wherein the initiator is a mixture of ammonium persulfate and N,N,N′,N′-tetramethylethylenediamine.

7. The method of claim 3, wherein the hydrophilic monomers have a mass percentage of about 69.3% to about 99% of a total mass of the gel precursor, the cross-linking agent has a mass percentage of about 0.45% to about 12.8% of the total mass of the gel precursor, and the initiator has a mass percentage of about 0.36% to about 19.5% of the total mass of the gel precursor.

8. The method of claim 1, wherein the peroxidase is horseradish peroxidase.

9. The method of claim 1, wherein the color developing agent is selected from a group consisting of potassium iodide, 3-hydroxy-2,4,6-tribromobenzoic acid/4-aminoantipyrine, o-phenylenediamine, and tetramethylbenzidine.

10. The method of claim 1, wherein the photo-degradable nanoparticles are prepared by:
mixing photodegradable acrylate and polyethylene glycol dimethacrylate to form a water phase solution;
mixing ammonium persulfate and N,N,N′,N′-tetramethylethylenediamine to form an oil phase solution;
stirring the oil phase solution and adding the water phase solution in the oil phase solution being stirred to form water-in-oil emulsion; and
filtering the water-in-oil emulsion to obtain the photo-degradable nanoparticles.

11. The method of claim 10, wherein the water phase solution and the oil phase solution are in a ratio selected from 1:16 to 1:105 by volume.

12. A biosensor for detecting glucose level comprising:
a gel substrate;
wherein glucose oxidase, peroxidase, and a color developing agent are dispersed in the gel substrate, the gel substrate defines a plurality of pores each having a diameter of about 50 nm to about 550 nm.

13. The biosensor of claim 12, wherein the peroxidase is horseradish peroxidase.

14. The biosensor of claim 12, wherein the color developing agent is selected from a group consisting of potassium iodide, 3-hydroxy-2,4,6-tribromobenzoic acid/4-aminoantipyrine, o-phenylenediamine, and tetramethylbenzidine.

15. The biosensor of claim 12, wherein the gel substrate is made of hydrogel or silicone hydrogel.

16. A method for detecting glucose level comprising:
providing a biosensor, the biosensor comprising a gel substrate, glucose oxidase, peroxidase, and a color developing agent dispersed in the gel substrate, the gel substrate defining a plurality of pores each having a diameter of about 50 nm to about 550 nm;
contacting the biosensor to a sample, thereby causing glucose in the sample to react with oxygen in an ambient environment under a function of the glucose oxidase to form hydrogen peroxide, and the hydrogen peroxide to oxidize the color developing agent under a function of the peroxidase, so that the color developing agent changes from colorless to a colored agent and causes the biosensor to display color; and
comparing the color of the biosensor with a color reference card to determine the glucose level in the sample.
